(19) United States
(12) Patent Application Publication
(10) Pub. No.: US 2023/0398359 A1
(43) Pub. Date: Dec. 14, 2023

(54) RECOVERY OF SENSORIMOTOR FUNCTION WITH NEUROPROSTHETIC SYSTEM AND METHOD THEREOF

(71) Applicants: Igor Aleksandrovich LAVROV, Saint Petersburg (RU); Maxim Olegovich TALANOV, Kazan (RU); Aleksandr Sergeevich TOSHCHEV, Kazan (RU); Andrey Pavlovich KUPTSOV, Kazan (RU)

(72) Inventors: Igor Aleksandrovich LAVROV, Saint Petersburg (RU); Maxim Olegovich TALANOV, Kazan (RU); Aleksandr Sergeevich TOSHCHEV, Kazan (RU); Andrey Pavlovich KUPTSOV, Kazan (RU)

(21) Appl. No.: 18/033,906

(22) PCT Filed: Jan. 18, 2021
(86) PCT No.: PCT/RU2021/000018

§ 371 Date: Apr. 26, 2023

(30) Foreign Application Priority Data
Nov. 2, 2020 (RU) 2020135886

(51) Int. Cl. A61N 1/36; G16H 50/50

(52) U.S. Cl. CPC A61N 1/36139; A61N 1/36003; A61N 1/36062; A61N 1/36125; G16H 50/50; A61N 1/36171; A61N 1/36103

(57) ABSTRACT

The present disclosure relates to a neuromorphic prosthesis system for facilitating sensorimotor functions in a subject in need thereof. The system incudes a plurality of stimulators having one or more channels configured to be disposed at various locations of the subject to provide an electrical stimulation of muscles groups, peripheral nerves, plexuses and/or spinal cord. The system further includes a plurality of sensors configured to detect and communicate a continues or cyclic distribution of a body weight, an angle joint motions, kinetics and/or electrophysiological parameters data and a plurality of controllers configured to receive a combined data from the plurality of sensors. The controllers are also configured to process the combined data and communicate the combined data to an fMEP-based artificial circuitry device, the fMEP-based artificial circuitry device configured for implementation of a reconstruction of topology of central pattern generator (CPG). The fMEP-based artificial circuitry device is configures to apply a bio-plausible neuronal topology for a motor function pattern and coordinates application of the electrical signals via the plurality of stimulators to the subject in need thereof to perform the motor function pattern.

### BACKGROUND

The present invention generally relates to systems for activating the sensorimotor function. More specifically, the present invention relates to a system for recovery from locomotion impairment after a spinal cord injury based on real-time evaluation of functional motor evoked potentials (fMEP) and fMEP-based reconstruction of a neuronal circuit topology, as well as a method for reconstruction of a neuronal circuit topology.

A spinal cord injury (SCI), brain injury, stroke and other neurological conditions interrupt the communication between the spinal cord and the rest of the body, thereby affecting voluntary control over function below the injury level. It has been shown in animal and human studies that epidural and/or intra-spinal electrical stimulation allows for activation of spinal locomotor circuits after SCI. For example, epidural electrical stimulation (EES) has restored coordinated locomotion in animal models with SCI, and isolated leg movements in individuals with motor paralysis.

It has been demonstrated that the structure of a spinal cord includes various microcircuits. These microcircuits process sensory information arising from movement of the limbs and inputs from various brain regions to produce locomotion responses and coordinate complex motor patterns. The pattern formation circuitry, responsible for rhythmic motor outcomes, like locomotion, is expected to include the set of nuclei with reciprocal inhibition informing a cyclic inhibitory and/or excitatory activity. It has been shown that the simulation of the microcircuit motifs can advance the development of neuromodulation solutions at different levels of the central nervous system. In particular, an implementation of the microcircuit motifs responsible for locomotion into neuroprosthetic systems to compensate for lost motor function can result in a restoration of motor functions in SCI patients.

### SUMMARY

In one aspect, the present invention provides a neuromorphic prosthesis system for facilitating sensorimotor function in a subject in need thereof. The system includes a plurality of stimulators having one or more channels configured to be disposed at various locations of the subject to provide coordinated electrical stimulation of muscle groups, peripheral nerves, plexuses, and spinal cord stimulation. The system further has a plurality of sensors configured to detect and communicate continuous or cyclic distribution of body weight, angle joint motions, kinetics, and electrophysiological parameters. In addition, the system includes controllers configured to receive data from the plurality of sensors and configured to process the combined data and communicate the combined data to a plurality of controllers configured to receive a combined data from the plurality of sensors and configured to process the combined data and communicate the combined data to an fMEP-based artificial circuitry device, the fMEP-based artificial circuitry device configured for implementation of a reconstruction of topology of central pattern generator (CPG). The fMEP-based artificial circuitry device applies a bio-plausible neuronal topology for a motor function pattern and coordinates application of electrical signals via the plurality of stimulators, compatible with a biological neuronal stimulation, to the subject in need thereof to perform the motor function pattern.

In another aspect, the present invention provides a method for activation of motor functions in limbs by targeting several levels, such as muscles, peripheral nerves, plexuses, and central circuitry at the spinal cord level of a subject in need thereof. The method includes providing a plurality of stimulators having one or more channels configured to be disposed at various locations of the subject to provide coordinated electrical stimulation of muscle groups, peripheral nerves, plexuses, and spinal cord stimulation by an electrical stimulation to innervate a plurality of targets. The method further includes providing a plurality of sensors configured to detect and communicate a continuous or cyclic distribution of body weight, angle joint motions, kinetics, and electrophysiological parameters data from the plurality of sensors. In addition, the method includes providing controllers configured to receive data from the plurality of sensors to process a combined data and communicate the combined data to an fMEP-based artificial circuitry device, the fMEP-based artificial circuitry device configured for implementation of a reconstruction of topology of central pattern generator (CPG). Further, the method includes stimulating the subject in need thereof to perform the motor function via electrical signals applied by the plurality of stimulators to the subject. The fMEP-based artificial circuitry device applies a bio-plausible neuronal topology for the motor function pattern and coordinates application of the electrical signals.

In yet another aspect, the present invention provides a method for reconstruction of a neuronal topology responsible for activation of motor functions in limbs based on evaluation of functional motor evoked potentials. The method includes detecting a neuronal response pattern in correlation with input stimulation parameters, analyzing the neuronal response pattern for a stable neuronal response pattern, the stable neuronal response pattern is determined based on a plurality of peaks, each peak having a maximum value and a minimum value, and each peak having amplitude and time. The method further includes evaluating the neuronal circuit topology by comparing the neuronal response pattern with a known neuronal biological response that forms an output signal to activate motor function in limbs.

In yet another aspect of the present invention, an oscillator motif (OM) is used for generating the neuronal response pattern, where the OM includes reciprocal excitation component and feedback inhibition component.

In yet another aspect of the present invention, a multi-level spinal locomotor circuit (mSLC) model is used for generating the neuronal response pattern, the mSLC model includes a monosynaptic layer and a polysynaptic layer.

### BRIEF DESCRIPTION OF THE DRAWINGS

In order that the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, aspects of the invention will be described and explained with additional specificity and detail through the use of the accompanying drawings.

FIGS. 1(A)-1(C) depict an oscillator motif (OM) according to embodiments of the invention;

FIG. 2 depicts a the multi-level spinal locomotor circuit (mSLC) model according to embodiments of the invention;

FIG. 3 depicts myograms of functional motor evoked potential (fMEP) patterns in response to an epidural electrical stimulation (EES) pulse for extensor (Soleus) and flexor (Tibialis anterior) muscles according to embodiments of the invention;

FIG. 4 depicts a detected neuronal response pattern based on a peak concept according to embodiments of the invention;

FIG. 5 depicts a polysynaptic fMEPs pattern having a pyramidal nature of peaks density according to embodiments of the invention;

FIG. 6 illustrates a latency being identified as the time (distance) between mono and poly fMEPs according to embodiments of the invention;

FIG. 7 depicts a neuromorphic prosthesis system according to embodiments of the invention;

FIG. 8 depicts a digital neuromorphic prosthesis system according to embodiments of the invention;

FIG. 9 depicts a spiking (analog) neuromorphic prosthesis system according to embodiments of the invention;

FIG. 10 illustrates a variety of spiking activity according to embodiments of the invention;

FIG. 11 illustrates shapes of spike in (a1) Izhikevich model, (a2) leaky integrate and fire model, and (a3) simple digital neuron; and

FIG. 12 depicts a simplified digital neuron model according to embodiments of the invention.

### DETAILED DESCRIPTION

Reference to “a specific embodiment” or a similar expression in the specification means that specific features, structures, or characteristics described in the specific embodiments are included in at least one specific embodiment of the present invention. Hence, the wording “in a specific embodiment” or a similar expression in this specification does not necessarily refer to the same specific embodiment.

Hereinafter, various embodiments of the present invention will be described in more detail with reference to the accompanying drawings. Nevertheless, it should be understood that the present invention could be modified by those skilled in the art in accordance with the following description to achieve the excellent results of the present invention. Therefore, the following description shall be considered as a pervasive and explanatory description related to the present invention for those skilled in the art, not intended to limit the claims of the present invention.

Reference to “an embodiment,” “a certain embodiment” or a similar expression in the specification means that related features, structures, or characteristics described in the embodiment are included in at least one embodiment of the present invention. Hence, the wording “in an embodiment,” “in a certain embodiment” or a similar expression in this specification does not necessarily refer to the same specific embodiment.

A system for recovery from locomotion impairment after a spinal cord injury (SCI) based on reconstruction of a neuronal circuit topology is provided. The structure of a spinal cord includes various neuronal microcircuits. These neuronal microcircuits process sensory information, for example, arising from limbs movement and inputs from various brain regions to produce locomotion responses. The pattern formation for the locomotion is introduced by the set of nuclei usually with reciprocal inhibition and cyclic inhibitory and/or excitatory projections and activity. The reconstruction and/or simulation of the neuronal microcircuit motifs can advance the development of neuromodulation solutions for enabling locomotion control in SCI patients.

The topology of the neuronal circuits responsible for generation of complex rhythmic patterns, central pattern generators (CPG), is largely undetermined. It has been shown, however, that epidural electrical stimulation (EES) can facilitate complex movements in SCI animals and patients by providing the sensory (e.g., electrical) input to the CPG circuits. Embodiments of the present invention provide that, as the topology of the CPG circuits mostly remains unknown, the fMEP-based reconstruction (i.e., reverse-engineering) of the neuronal circuitry of the spinal cord CPG, which is responsible for a motor pattern formation, allows for generating a computational model (in silico) that can be implemented in a single-board computer (in cyberico). The resulting in silico computational model of the neuronal topology can be then implemented in a CPG implementation device that allows for the generation of the electrical signals compatible with the biological neuronal stimulation of the muscles of a SCI patient to activate motor functions in limbs of a SCI patient.

For purposes of reconstruction of the neuronal topology responsible for the motor patterns of a SCI patient, according to embodiments of the present invention, a neuronal response pattern is detected based on known inputs (i.e. sensory modulation and parameters of stimulation) and electrophysiological outputs in correlation with specific pattern. The neuronal response pattern is then analyzed in relation to the time and amplitude modulation of responses. Based on the analysis of the neuronal response pattern, neuronal microcircuit topology is reconstructed. The neuronal microcircuit topology is then implemented in a pattern formation circuitry that can generate an electrical signal compatible with the biological neuronal stimulation to activate motor function in a limb.

Detection of the Neuronal Response Patterns in Correlation with Input Stimulation

According to embodiments of the present invention, the neuronal response pattern is detected based on known or partially known parameters of stimulation, such as, amplitude, frequency and duty cycle of an isolated neuronal circuitry. The following in vivo models can be used for to evoke the motor responses: AIR stepping (i.e., a biological model where a subject performs steps in air without touching a treadmill with full support from body weight support (BWS) system), a model where a subject barely touches the treadmill and performing steps (TOE), a biological model where a subject performs the bipedal step with support of BWS only to maintaining vertical stance (PLT (plantar)). In addition, biological model having different treadmill speeds, models that included pharmacological influence by quipazine (QPZ) and strychnine (STR), and models with various known epidural stimulation can be used to detect the neuronal response patterns. Variety of inputs and outputs assessed in experimental in vivo settings was described in detail elsewhere [Islam et al., 2019]. We have implemented this approach to evaluate the circuitry potential topology and functional organization based on variation in motor outputs on a variety of sensory inputs. In order to record evoked motor responses any known method can be used, for example, capturing electromyography (EMG) by placing steel wires on the following muscles gluteus maximus (GLU); rectus femoris (RF); vastus lateralis (VL); tibialis anterior (TA); soleus (SOL); and medial gastrocnemius (MG).

Further, in silico computational models can be used to detect and simulate the neuronal response patterns to the known input parameters, for example, NEURON, NEST and/or GRAS simulators. According to one embodiment, the Hodgkin-Huxley model of a neuron can be used with NEURON simulator. See Hodgkin, A. L., Huxley, A. F.: A quantitative description of membrane current and its application to conduction and excitation in nerve. The Journal of Physiology 117(4), 500, 544 (1952). NEST simulator can also be used. NEST is a simulator for spiking neural network models that focuses on the dynamics, size and structure of neural systems rather than on the exact morphology of individual neurons. See Jordan, J., Ippen, T., Helias, M., Kitayama, I., Sato, M., Igarashi, J., Diesmann, M., Kunkel, S.: Extremely scalable spiking neuronal network simulation code: From laptops to exascale computers. Frontiers in Neuroinformatics 12, 2 (2018).

GRAS simulator employs the GPU processing of bio-plausible neuronal networks using C++ programming language and Nvidia CUDA technology. More specifically, the GRAS simulator is a real-time simulator of neuronal activity with predefined neuronal topology. It can support three different neuron models: (1) Even Simpler Real-Time Model of Neuron (ESRN), see Leukhin, A., Talanov, M., Suleimanova, A., Toschev, A. & Lavrov, I. Even simpler real-time model of neuron. Bio-NanoScience 1-4 (2020); (2) Hodgkin-Huxley neuron, see Hodgkin, A. L. & Huxley, A. F. A quantitative description of membrane current and its application to conduction and excitation in nerve. The J. Physiol. 117, 500-544; (3) Izhikevich neuron, see Izhikevich, E. M. Polychronization: Computation with spikes. Neural Comput. (2006). GRAS focuses on the whole neuronal topology model in contrast to the individual neurons in NEST. GRAS also uses GPU processing for its calculations due to its high level of efficiency.

According to embodiments of the present invention, to detect and simulate the neuronal response patterns, a simplified simulator model of a neuron (Even Simpler Real-Time Model of Neuron (ESRN)) can be used for enabling a real-time processing in an embedded bio-compatible device. One of critical problems of a bio-plausible neuro-simulation is the time of computation wearable neuro-prosthesis require real-time processing for the topology of thousands of neurons and hundreds of thousands of synapses on a single board computer with limited weight and usually no connection to an outside network. The simplified digital neuron model corresponds to the following requirements: a real-time processing on a single-board computer, takes into account only bio-plausible spike times, refractory period, inhibitory influence, and threshold-based processing of the inbound spikes. According to one embodiment of the present invention, the simplified simulator model includes the following assumptions to optimize the code of the neuronal model for the real-time processing of a large number of neurons (thousand and more) and a large number (hundreds of thousands or more) of synapses: only motor neurons and interneurons are modeled and amplitude of spikes is not considered, but rather bio-plausible spike and refractory period duration is utilized. In order to perform the computations in real-time the following simplified equation for the membrane potential is used

L=ΣW±leakage+noise

where the level is the sum of projection weights, leakage, and noise.

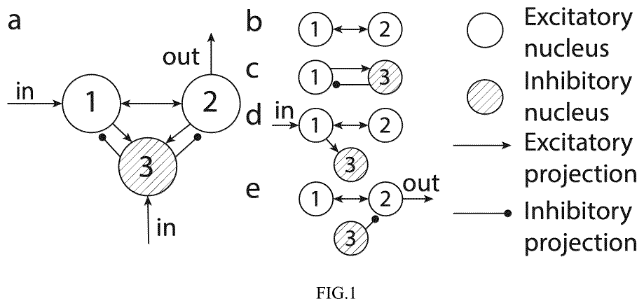

According to embodiments of the present invention, in the neuronal response pattern detection and analysis, an oscillator motif (OM) as shown and described in FIGS. 1(A)-1(C), is used as a foundation (building blocks) of the neuronal circuits and their microcircuit structure found, for example, in the spinal cord. The OM includes four frequent motifs of the central nervous system (CNS): (i) feed-back inhibition; (ii) rhythmic excitation; (iii) divergence; and (iv) convergence. More specifically, the OM consists of two functional components: (i) reciprocal excitation (1, 2) (shown in FIG. 1(B)) and (ii) feedback inhibition 1, 3 (shown in FIG. 1(C)). The OM can generate neuronal activity of different durations. The duration of the neuronal activity depends on a balance of weights between the excitatory and inhibitory nuclei as shown in FIGS. 1(A)-1(C). The input nucleus 1 receives signals, for example, from afferents and/or EES, and triggers reciprocal excitation with a second nucleus 2. The first and second excitatory nuclei 1, 2 have strong reciprocal projections to produce output activity that is terminated by the inhibitory projection from a third nucleus 3, which has weak projections to excitatory nuclei 1, 2. Additionally, the output activity could be terminated by an external excitatory stimulus of the third nucleus (as shown in FIG. 1A). In other words, according to embodiments of the present invention, the OM generates the neuronal activity with predefined duration and amplitude via balance between the inhibitory and excitatory projections.

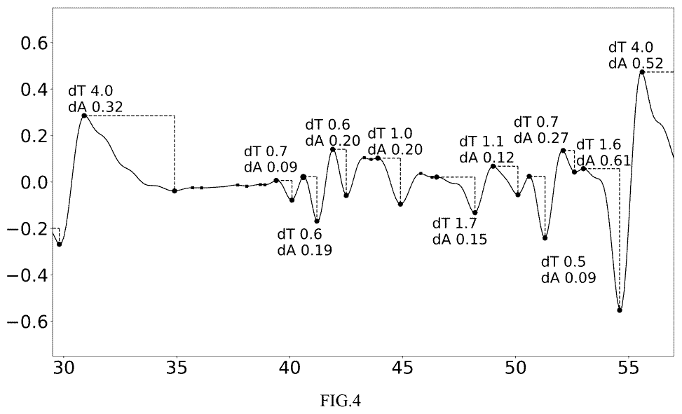

As shown in FIGS. 1(A)-1(C), the OM creates neuronal activity dynamics of the functional motor evoked potential (fMEP). fMEP can be measured, for example, through (i) the latency after each EES pulse; (ii) the duration, (iii) the amplitude, and (iv) the number of peaks between the pair of maximum and minimum extrema (as shown in FIG. 4).

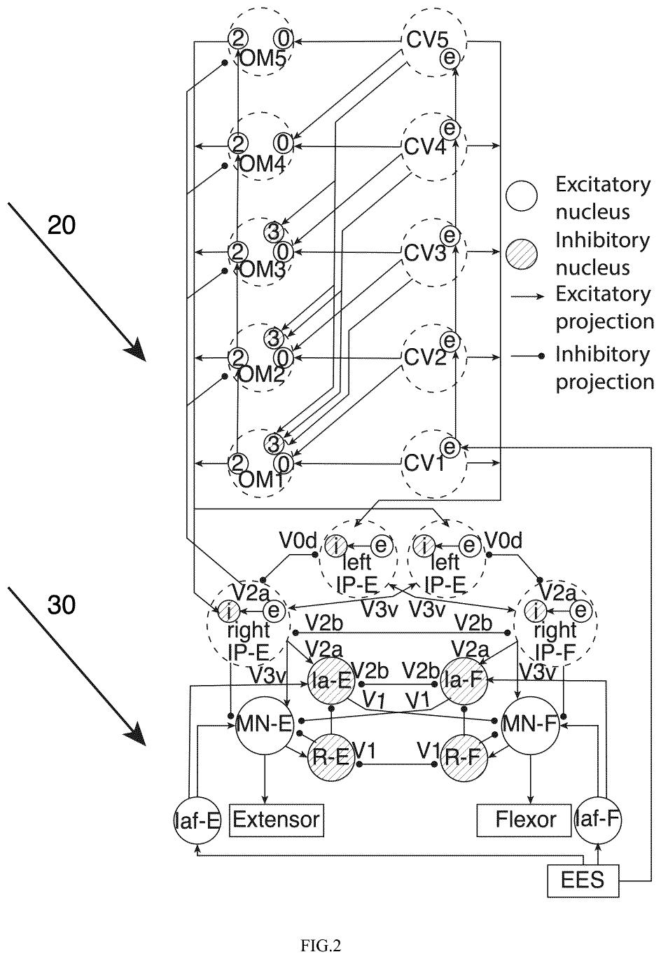

FIG. 2 shows the detailed diagram of the multi-level spinal locomotor circuit (mSLC) model according to embodiments of the present invention. During stepping, fMEPs generation is triggered by the sequential activation of the monosynaptic and polysynaptic layers via combined inputs from cutaneous ventral (CV) sensory afferents and EES pulses thereby amplifying the neuronal activity of the CVs 1 through 5 (as shown in FIG. 2). The mSLC includes polysynaptic level 20 (associated with poly fMEPs) and monosynaptic level 30 (associated with mono fMEPs). The monosynaptic level 30 includes motoneurons (MN-E, MN-F), afferents fibers (Iaf-E, Iaf-F), interneurons that are associated with Ia-afferents, inhibit motoneurons (Ia-E, Ia-F) of antagonist-muscle and Renshaw cells (R-E, R-F), which save muscle from strong contractions forming negative feedback. As shown in FIG. 2, this level is triggered by Ia-afferents activated by, for example, EES. The polysynaptic level 20 forms polysynaptic responses that are usually longer than monosynaptic, but have lower maximal amplitude, thereby creating complex neuronal structures. Polysynaptic level 20 consists of five layers shown in FIG. 2.

The number of layers of the polysynaptic level 20 corresponds to the number of polysynaptic fMEP delays. Each of the five layer contains its own OM (as shown in FIG. 1) and is formed via two motifs: reciprocal excitation (FIG. 1(B)) and feedback inhibition (FIG. 1(C)), that in turn contains the inhibitory nucleus which adjusts the length of neuronal activity of the OM and guarantees the timely generation of neuronal output. That is, polysynaptic level 20 includes, as illustrated in FIG. 2, CV 1 through 5, OM 1 through 5, the excitatory (e) and inhibitory (i) parts of inter-neuronal pool: IP-E, IP-F, where upper case letter E stands for nuclei of extensor muscle, F—flexor. Each CV is the set of ascending fibers that includes fibers activated by a CV input and forming the baseline activity of corresponding CV and activated by, for example, the EES enhancement of the fibers electrical activity (e) nuclei.

The increased delay between the monosynaptic and the polysynaptic responses is formed due to the inhibition of the lower layers by the upper layers (as shown in FIG. 2), excitatory projections to an inhibitory nucleus. The higher CVs 3 through 5 inhibit lower OMs 1 through 3. Each layer is triggered by sensory afferents or cutaneous ventral CVs 1 through 5 and/or EES.

There are two modes of the polysynaptic level extensor muscle mode. The first one is a stance mode, where each OM is triggered by co-activation from CVs 1 through 5 and EES. Cutaneous afferents are triggered based on the distribution of body weight toe to heel. In the second mode (swing mode), the polysynaptic layer 20 works sequentially independent of the CVs activation because a CV is not activated when a foot is in the air. This way the OM 1 (shown in FIG. 2) is triggered by EES and CV 1 of the opposite leg.

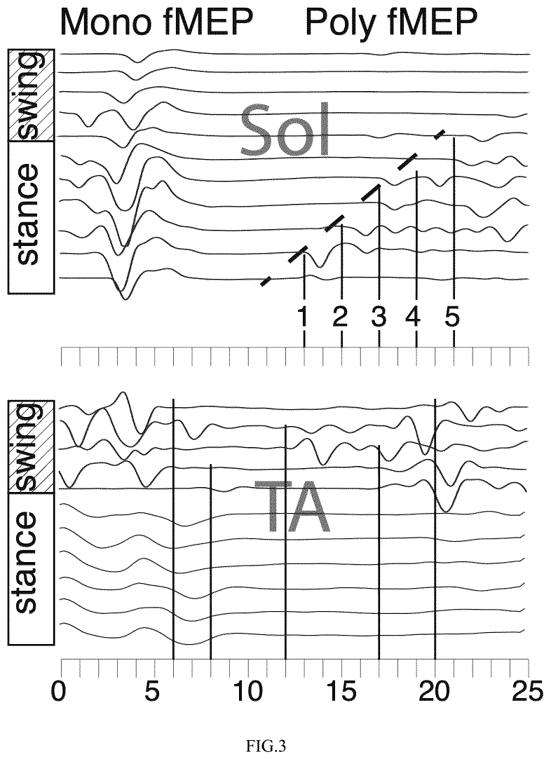

FIG. 3 shows myograms of fMEP patterns in response to an EES pulse for extensor (Soleus) and flexor (Tibialis anterior) muscles with marked latency for each slice (the period between the EES pulse stimulations of 40 Hz). During a stance mode, the first EES pulse and heel cutaneous afferents trigger the OM 1 via CV 1 neuronal groups (as shown in FIG. 2). This produces the neuronal activity of the first slice (13 to 15 millisecond (ms)), as shown in FIG. 3. The next EES pulse and the body weight transferred from the heel towards the toe activates groups of neurons in CV 1 and CV 2 and triggers (via CV's projections) OM 1 and OM 2, thereby generating the extended fMEP with higher amplitude of the next slice (13 to 21 ms). The third EES pulse and the weight transfer activates the CV 3 that triggers OM 2 and later OM 3 that generate long fMEP (15 to 23 ms) and fMEP (0 to 3 ms) of the fourth slice. The CV3 inhibits the OM 1 implementing feed-back inhibition.

The fourth and fifth EES pulses and weight transfers, close to the toe cutaneous afferents projections to CV 4-OM 3 and OM 4 (19 ms) that create the short and intensive activity of the slices four and five (19 to 25 ms and 0 to 3 ms, respectively) of the fifth and sixth slices and inhibit OM 1 and OM 2. The sixth EES pulse has special function as it triggers the OM 5 via CV 5 jointly with weight transfer to the toe, and inhibits OM 1 through OM 3 (as shown in FIG. 2), thereby effectively preventing them from generating early responses and projects the short neuronal activity with relatively high amplitude.

During the swing mode, given that there is no cutaneous input, three delays are shown in FIG. 3, namely 17 ms, 12 ms and 20 ms, and durations of 17 ms, 44 ms and 5 ms. The five layer structure shown in FIG. 2 produces the flexor fMEP pattern shown in myogram of FIG. 3. More specifically, the first EES pulse via CV 1 triggers OMs of the first and second layers. First the OM 1 is triggered and the 0 prior to 1 excitatory nucleus of the OM 1 (as shown in FIG. 2) tigers the second nucleus of the OM 2. This produces strong fMEP activity starting from 17 ms of the slice seven and till 3 ms of the slice eight (shown in FIG. 3TA). The second EES pulse triggers CV 2 along with CV 1 that effectively enhances the output fMEP from 17 ms to 25 ms of the slice 8 ms until 3 ms of the slice nine (shown in FIG. 3TA). The third and forth EES pulses trigger OM 3 via CV 3 and CV 4, and nuclei 2 of OM 3 triggers the OM 4 (as shown in FIG. 2). This pair produces long and intensive fMEPs starting from 12 ms of the ninth slice until 6 ms of the eleventh slice (shown in FIG. 3TA). Then, the fifth EES pulse triggers CV 5 and the projection from nucleus 0 the OM 4 to the nucleus 0 of the OM 5 and activates the OM 5 (as shown in FIG. 2). This produces neuronal pulses from 20 ms to 25 ms of the eleventh slice.

The flexor mode is organized in a sequential OM activation manner when the second nuclei triggers the next OMs (as shown in FIG. 2). The IP-E Right and IP-E Left has inhibitory presynaptic projection that inhibits excitatory projections of the second nuclei of OMs during the stance mode, which are activated via CV 1 through 5. During the swing mode those projections play an important role in flexor pattern formation.

According to embodiments of the present invention, there are two main input stimulation mSLC models used for the detection of the neuronal response patterns EES and cutaneous. The main impact of the cutaneous input is applied to the monosynaptic level 30 that works simultaneously for both right and left legs. The balancing between two legs has diagonal nature, when the right extensor is activated due to cutaneous input the flexor of the left leg is also active. On the other hand when the extensor of the left leg is active, the flexor of the right leg is activated and the rest is inhibited.

### Analysis of the Detected Neuronal Response Patterns

According to embodiment of the present invention, the analysis of the detected neuronal response pattern described above is based on an evaluation of the functional motor evoked potentials (fMEP) and analysis of the complex patterns produced by the neuronal network. In particular, a peak is considered to be the pair of extrema: first maximum and later minimum as it is depicted in FIG. 4. The delta (Δ) between the maximum values and minimum values is the amplitude of the peak, the duration of the peak=tₘᵢₙ−tₘₐₓ (as shown by dA in FIG. 4). According to embodiments of the present invention, to be used as a peak the peak duration should be in the range between 0.15 ms and 4 ms and the relative amplitude should be greater than 3% of the maximal fMEP amplitude as shown in FIG. 4. The lowest time and amplitude thresholds can be determined by taking a fifteenth and twenty fifth percentile of all peaks and comparing different time and amplitude thresholds. The ranges described herein allow avoiding long myogram waves which are not peaks or “noise-like” peaks.

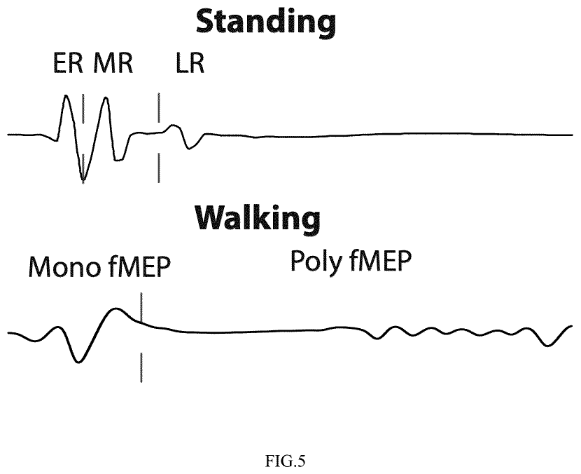

According to embodiments of the present invention, the myograms are analyzed to determine stable patterns by any suitable method, for example, kernel density estimation (KDE). As shown in FIGS. 3 and 5, using in vivo and in silico data from the various models, for example, AIR, TOE, PLT, QPZ, STR, NEURON an/or GRAS, a stable pattern can be identified with a specific number of peaks, relative aggregated amplitude, and latency.

According to embodiments of the present invention, the data can be validated by KDE test. In particular, the validation of in silico data via in vivo myograms requires the unified representation of datasets. The in vivo data or myogram packs are recorded in a biological model (animal) during its motion that by itself potentially creates unwanted artifacts, for example, a zero point shift that is unacceptable in the context of in silico data validation because the simulation data does not include it as the in silico model does not have a shift of electrodes during the motion. To compensate for the zero point shift, the PCA eigenvectors can be used to rotate a center points of myogram packs in in silico model.

According to embodiments of the present invention, in light of the different nature of the polysynaptic and monosynaptic fMEPs, two different processes of the analysis of the neuronal response patterns can be used. First, the monosynaptic fMEPs indicate the regularity of peaks in the range of 3 ms to 8 ms on each slice of the in vivo or in silico data (as shown in FIG. 3). On the other hand, the polysynaptic fMEPs pattern has pyramidal nature of peaks density, for example, in PLT mode in the range of 8 ms to 25 ms and 0 ms to 3 ms of a successive slice (as indicated via KDE and shown in FIG. 5). The borderline between mono and poly fMEPs is selected via analysis of the density of peaks that do not fall into the mono fMEP interval, for example, in the range of 8 ms to 25 ms.

Further, the latency A between responses identifies the presence of more than one nuclei generating responses and its value identifies the number of synapses between activation of each nucleus, taking into account one synaptic delay of 2 ms. The number of peaks identifies the distribution of latencies and projection weights in a nucleus (as shown in FIG. 5). The A of the relative amplitude indicates the presence of more than one nuclei with different neuronal activity (as shown in FIG. 5). A response duration is correlated with the number of peaks and identifies the distribution of latencies and if the overall duration is longer than one synaptic delay the presence of internal nucleus topology with several synaptic connections.

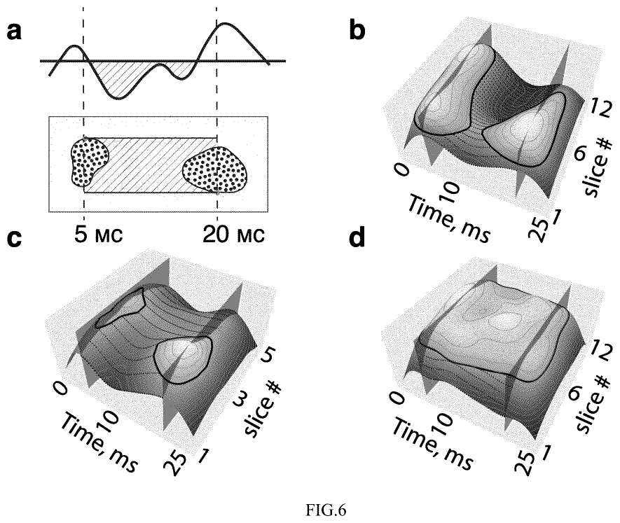

As illustrated in FIG. 6, a latency is identified as the time (distance) between mono and poly fMEPs in the time frame between 5 ms and 20 ms where there is an overlap with mono fMEPs that are between 3 ms and 8 ms and guaranteed poly fMEPs that are between 8 ms to 25 ms. To detect and measure the overall value of latency of each fMEP pack of each in vivo model in each mode, 3D KDE map approach with the comparison of volumes below the threshold isoline can be used. The threshold isoline is determine by the following formula for QPZ/STR model:

isolineₜₕ=⅔(max+min)

and for AIR/TOE/PLT models:

isolineₜₕ=½(max+min)

where max is the maximal value of peak density and min is the minimal value of peak density in the current fMEP pack.

According to embodiments of the present invention, the neuronal response pattern can be further validated taking into account the low level details of mono and poly fMEPs' latency volume between mono and poly fMEPs, peaks number, amplitudes in mono and poly fMEPs, as well as a shape of polysynaptic patterns (shown in FIG. 3) by any suitable method, such as KDE test.

### Reconstruction of the Neuronal Circuit Patterns/Topology

According to embodiments of the present invention, the results described above, such as (i) the approximate number of nuclei (ii) the approximate latencies between activation of each nucleus (ii) the distribution range of weight and latencies of each projection (synaptic connection), and (iv) the types of neurons for each nucleus are used to reconstruct the neuronal circuit topology with known types of neurons that form an output signal. According to embodiments of the present invention, two levels of abstraction can be used for reconstruction of the neuronal circuit topology, a high-level topology and a low-level topology. In particular, the high-level topology of nuclei includes the latency between neuronal responses, the relative amplitude and number of peaks of each response. The duration, aggregated amplitude and the number of peaks of response, the distribution of projection weights and latencies within one synaptic delay is determined to reconstruct the low-level topology. For example, if a duration exceeding one synaptic delay the neuronal topology within a nucleus is assumed, taking into account the number of neurons per nuclei and distribution of their connection parameters. Then, using the timings of a nucleus activation taking into account inbound activity of the co-activation projections of a nucleus is determined. In addition, it is possible to use clustering to identify neurons that produced the spike in case of high sampling rate of the neuronal activity recording. When applying the forgoing approaches, a neuronal topology can be reconstructed using known motifs of neurons in, for example, the CNS. The neuronal microcircuit topology can then be implemented in a CPG device that can generate an electrical signal compatible with the biological neuronal stimulation to activate motor function in a limb.

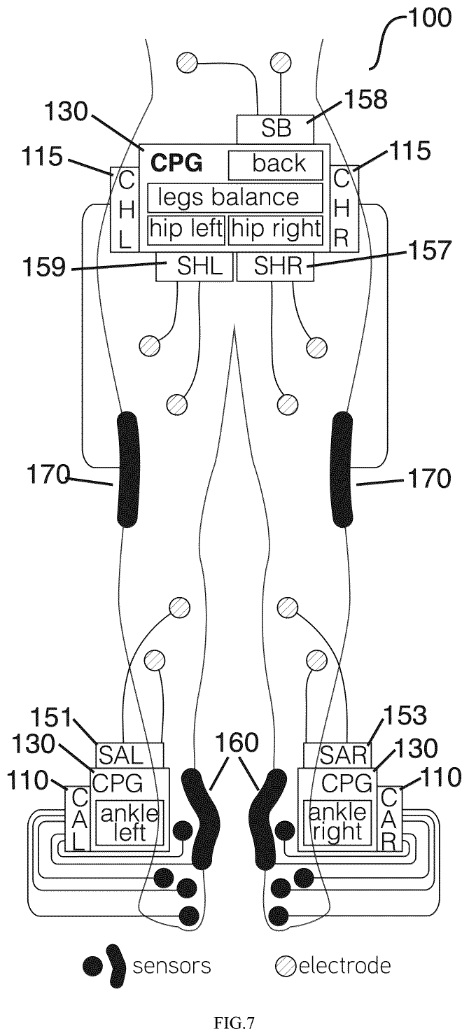

FIG. 7 illustrates a neuromorphic prosthesis system 100. According to embodiments of the present invention, the neuromorphic prosthesis system 100 is configured to facilitate walking in a patient with a complete or partial SCI and paralyzed legs. The neuromorphic prosthesis system 100 includes, for each limb, an ankle controller 110 and a hip controller 115, a central pattern generator device 130, and stimulators 150. The controllers 110, 115 are data processing apparatuses that receive an input data from the pressure sensors 160 that can be positioned on the insole and the flex sensors 170 that can be positioned around the ankle and/or knee. The ankle controllers 110 can be positioned around the ankles and transmit data to the hip controllers 115. The hip controllers 115 preprocess the data received from the ankle controllers 110 and combine the received data from the ankle controllers 110 with the data that the hip controllers 115 received from the flex sensors 170. Then, the hip controllers 115 transmit the combined data to the central CPG implementation device 130.

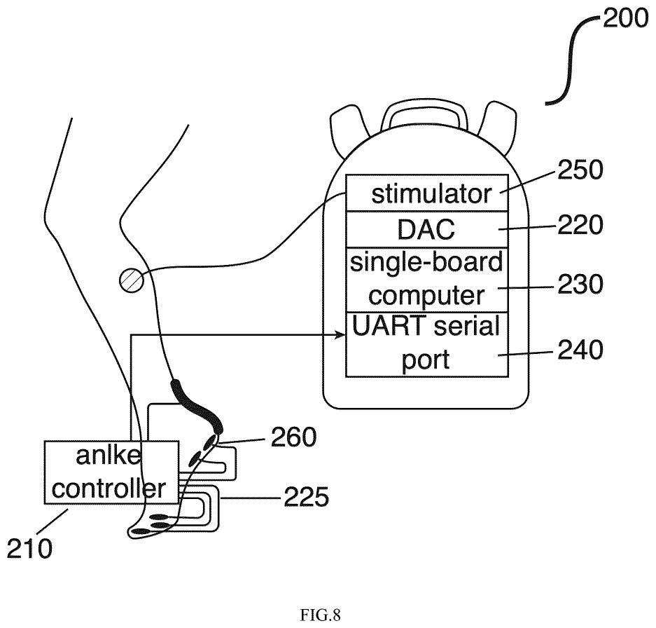

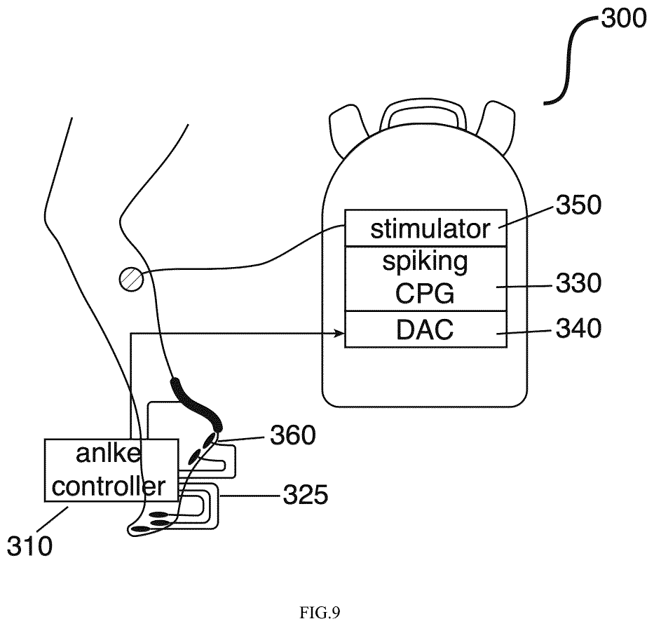

The central CPG device 130 can be implemented as a synchronized set of one board digital computers (for example, PINE A 64) (as shown in FIG. 8) or spiking (analog) schematics (as shown in FIG. 9), implementing the central pattern generator of a segment of a spinal cord topology or an application-specific integrated circuit (ASIC) chip with the same functionality. The CPG 130 device implements the bio-plausible topology of a walking pattern generator that is capable of the generation of the electrical signals compatible with the biological neuronal stimulation of muscles of lower limbs. The neuronal topology of the CPG is described in details in the FIG. 2 as the two levels of topology (monosynaptic and polysynaptic) of the locomotion pattern generator with the five layers of the polysynaptic level of the locomotion pattern generator. The CPG device 130 can also balance flexor-extensor muscle pairs and left and right legs.

Stimulators 150 can include a back stimulator 158, a left ankle stimulator 151, a right ankle stimulator 153, a right hip stimulator 157, and a left hip stimulator 159. The stimulators 150 can be electrodes positioned at the muscles, nerves, plexuses or for transcutaneous spinal cord stimulation. Stimulators 150, 250 can be single or multichannel stimulators suitable for the stimulation according to embodiments of this invention.

The output of the CPG triggers the stimulators 151, 153, 157, 158, 159 that innervate the hind limb muscles (hips and feet) via ascending nerves or directly, thereby transmitting the walking pattern individually to each flexor and extensor muscle pair. The muscles in their turn balance the weight towards the locomotion direction that changes the weight distribution registered by pressure sensors 160 and angles of joints registered by flex sensors 170.

The system 100 can be an open-loop or closed-loop feedback system. In some embodiments, controllers 110, 115 can communicate with an external computing system (not shown). For example, a user may define a stimulation program on the external computing system, which can then be uploaded to the controllers 110, 115 or, alternatively, to the central CPG implementation device 130.

According to another embodiment of the present invention, a digital neuromorphic prosthesis system 200 is shown on FIG. 8. The digital neuromorphic prosthesis system 200 includes an ankle controller 210 and an insole controller 225, a CPG device 230, a stimulator 250, pressure sensors 260 a digital-to-analog converter (DAC) 220 and a universal asynchronous receiver-transmitter (UART) serial port 240. According to this embodiment, the CPG device 230 can be a single-board computer.

Here, input signals are formed through the weight distribution “toe-to-heel” and are processed by the insole controller 225. From the insole controller 225 the pressure sensors data is transmitted via the UART serial port 240 to the central pattern generator device 230 (single-board computer) where the weighted A of the sensors data “toe-to-heel” is used as the input for the CPG device 230 implementation. The CPG device 230 generates the digital locomotor patterns in real-time for the hind-limb muscles and triggers the stimulator 250 via the DAC 220. That is, the DAC 220 converts stimulators 250 voltage signals to the current signals in the safe for the human nerve/muscle stimulation range, for example, from −100 to +100 mA.

According to yet another embodiment of the present invention, a spiking (analog) neuromorphic prosthesis system 300 is shown on FIG. 9. The spiking neuromorphic prosthesis system 300 includes an ankle controller 310 and an insole controller 325, a spiking central pattern generator (CPG) device 330, a stimulator 350, pressure sensors 360, and a digital-to-analog converter (DAC) 340.

Here, input signals are formed through the weight distribution “toe-to-heel” and are processed by the insole controller 325. From the insole controller 325 the pressure sensors data is transmitted to the spiking CPG device 330 through DAC 340. The spiking CPG device generates the walking pattern with a defined frequency and duration which corresponds to a walking speed. The walking pattern is generated by the analog CPG device's 330 board, and is transmitted to the stimulator 350 that innervates the descending nerves to trigger the muscle.

### Example #1 (Oscillator Motif)

Abstract

In order to reconstruct the basic circuit motif of more complicated neural networks in the central nervous system (CNS) a new approach of the simulation of the oscillator motif is introduced. The microcircuit of the oscillator motif is built using the four frequent in the central nervous system motifs: feed-back inhibition, rhythmic excitation, divergence and convergence. The simulation indicates four basic neural activity generation modes with minimal weights of connections between four nuclei, maximal weights between nuclei, weights in between the maximal and minimal and the involvement of inhibitory nuclei that suppress the output neuronal activity effectively. To test the oscillator motif by comparing the generated activity, two neurosimulators have been used: NEURON, GRAS.

Introduction

We use one of the possible solutions to reconstruct the microcircuitry of the basic oscillator motif (OM) that could be used in the spinal cord central pattern generator (CPG), its justification and validation via neurosimulation using two simulators: NEURON (See Hines, M., Carnevale, N.: The NEURON simulation environment, 2nd edn. (2003); NEST (Jordan, J., Ippen, T., Helias, M., Kitayama, I., Sato, M., Igarashi, J., Diesmann, M., Kunkel, S.: Extremely scalable spiking neuronal network simulation code: From laptops t) o exascale computers. Frontiers in Neuroinformatics 12, 2 (2018)) and a new C++ programming language simulator GRAS.

Motifs Review

There are several works dedicated to the structure of a spinal cord describing microcircuits (see Ampatzis, K., Song, J., Ausborn, J., El Manira, A.: Separate Microcircuit Modules of Distinct V2a Interneurons and Motoneurons Control the Speed of Locomotion. Neuron 83(4), 934{943 (2014)); Chopek, J. W., Nascimento, F., Beato, M., Brownstone, R. M., Zhang, Y.: Subpopulations of Spinal V3 Interneurons Form Focal Modules of Layered Pre-motor Microcircuits. Cell Reports 25(1), 146156.e3 (2018); Deska-Gauthier, D., Zhang, Y.: The functional diversity of spinal interneurons and locomotor control. Current Opinion in Physiology 8, 99-108 (2019)), starting from the basic studies describing reflex arc (see Ilya A. Rybak, Kimberly J. Dougherty, Natalia A. Shevtsova: Organization of the Mammalian Locomotor CPG: Review of Computational Model and Circuit Architectures Based on Genetically Identifed Spinal Interneurons. eNeuro (2015); Markin, S. N., Klishko, A. N., Shevtsova, N. A., Lemay, M. A., Prilutsky, B. I., Rybak I. A.: Afferent control of locomotor CPG: insights from a simple neuromechanical model: Afferent control of locomotor CPG. Annals of the New York Academy of Sciences 1198(1), 21-34 (2010); Rybak, I. A., Shevtsova, N. A., Lafreniere-Roula, M., McCrea, D. A.: Modelling spinal circuitry involved in locomotor pattern generation: insights from deletions during fictive locomotion. The Journal of physiology 577(2), 617-639 (2006)) to most advanced approaches including multi-layer models of the rhythmic patterns generators (see, e.g., van den Brand, R., Heutschi, J., Barraud, Q., DiGiovanna, J., Bartholdi, K., Huerlimann, M., Friedli, L., Vollenweider, I., Moraud, E. M., Duis, S., Dominici, N., Micera, S., Musienko, P., Courtine, G.: Restoring Voluntary Control of Locomotion after Paralyzing Spinal Cord Injury. Science 336(6085), 1182-1185 (2012); Capogrosso, M., Wenger, N., Raspopovic, S., Musienko, P., Beauparlant, J., Bassi Luciani, L., Courtine, G., Micera, S.: A Computational Model for Epidural Electrical Stimulation of Spinal Sensorimotor Circuits. Journal of Neuroscience 33(49), 19326-19340 (2013); Moraud, E. M., Capogrosso, M., Formento, E., Wenger, N., DiGiovanna, J., Courtine, G., Micera, S.: Mechanisms Underlying the Neuromodulation of Spinal Circuits for Correcting Gait and Balance Deficits after Spinal Cord Injury. Neuron 89(4), 814-828 (2016); Wagner, F. B., Mignardot, J. B., Go-Mignardot, C. G. L., Demesmaeker, R., Komi, S., Capogrosso, M., Rowald, A., Se_a˜nez, I., Caban, M., Pirondini, E., Vat, M., McCracken, L. A., Heimgartner, R., Fodor, I., Watrin, A., Seguin, P., Paoles, E., Keybus, K. V. D., Eberle, G., Schurch, B., Pralong, E., Becce, F., Prior, J., Buse, N., Buschman, R., Neufeld, E., Kuster, N., Carda, S., Zitzewitz, J.v., Delattre, V., Denison, T., Lambert, H., Minassian, K., Bloch, J., Courtine, G.: Targeted neurotechnology restores walking in humans with spinal cord injury. Nature 563(7729), 65 (2018)). The formation of the locomotor pattern historically was explained by the set of nuclei usually with reciprocal inhibition and cyclic inhibitory or/and excitatory projections and activity (see, e.g., Ampatzis, K., Song, J., Ausborn, J., El Manira, A.: Separate Microcircuit Modules of Distinct V2a Interneurons and Motoneurons Control the Speed of Locomotion. Neuron 83(4), 934-943 (2014)); Chopek, J. W., Nascimento, F., Beato, M., Brownstone, R. M., Zhang, Y.: Subpopulations of Spinal V3 Interneurons Form Focal Modules of Layered Pre-motor Microcircuits. Cell Reports 25(1), 146156.e3 (2018)). At the same time the structure of pattern formation and organization of microcircuits capable of the locomotion pattern generation remains unclear (see, e.g., Gad, P., Lavrov, I., Shah, P., Zhong, H., Roy, R. R., Edgerton, V. R., Gerasimenko, Y.: Neuromodulation of motor-evoked potentials during stepping in spinal rats. Journal of Neurophysiology 110(6), 1311-1322 (2013). A recurrent stable neuronal microcircuits motifs (see English, D. F., McKenzie, S., Evans, T., Kim, K., Yoon, E., Buzs_aki, G.: Pyramidal Cell-Interneuron Circuit Architecture and Dynamics in Hippocampal Networks. Neuron 96(2), 505-520 (2017)) found in a brain or/and spinal cord (see, e.g., Chopek, J. W., Nascimento, F., Beato, M., Brownstone, R. M., Zhang, Y.: Subpopulations of Spinal V3 Interneurons Form Focal Modules of Layered Pre-motor Microcircuits. Cell Reports 25(1), 146156.e3 (2018)). During the analysis the following criteria have been used: (1) the generator with reciprocal excitation of at least two nuclei (2) it should be managed via the third nuclei that after an activity period inhibiting a neuronal activity of the generator (3) the whole motif could be self-regulated and self-organized via Spike-Timing-Dependent Plasticity (STDP) learning (see, e.g., van den Brand, R., Heutschi, J., Barraud, Q., DiGiovanna, J., Bartholdi, K., Huerlimann, M., Friedli, L., Vollenweider, I., Moraud, E. M., Duis, S., Dominici, N., Micera, S., Musienko, P., Courtine, G.: Restoring Voluntary Control of Locomotion after Paralyzing Spinal Cord Injury. Science 336 (6085), 1182-1185 (2012).

Method.

Considering the specifics of the generation of neuronal activity, the key low level component of complex circuitry, named here oscillator motif (OM) is proposed (FIG. 1a). Initial design of OM was inspired by works of Paz (Paz, J. T., Huguenard, J. R.: Microcircuits and their interactions in epilepsy: is the focus out of focus? Nature Neuroscience 18(3), 35-359 (2015); Womelsdorf (Womelsdorf, T., Valiante, T. A., Sahin, N. T., Miller, K. J., Tiesinga, P.: Dynamic circuit motifs underlying rhythmic gain control, gating and integration. Nature Neuroscience 17(8), 1031-1039 (2014)) and English (English, D. F., McKenzie, S., Evans, T., Kim, K., Yoon, E., Buzsaki, G.: Pyramidal Cell-Interneuron Circuit Architecture and Dynamics in Hippocampal Networks. Neuron 96(2), 505-520 (2017)) and is based on recurrent or rhythmic excitation of two nuclei (FIG. 1B), feed-back inhibition (FIG. 1c), divergence (FIG. 1d), and convergence (FIG. 1e). The basic function of the OM is to generate neuronal activity for the specified period from 2 ms to 15 ms. For this purpose, the rhythmic excitation motif is used that includes two nuclei and with reciprocal excitatory projections (FIG. 1B nuclei: 1, 2) that produce extended neuronal activity. For activity termination two feed-back inhibition motifs (FIG. 1c nuclei: 1, 3) are used with weak excitatory and strong inhibitory projections that determine the duration of neuronal activity. The inbound electrical activity, indicated as an arrow to the nucleus 1, triggers the reciprocal excitation of 1 and 2 that in turn provides output neuronal activity and weakly excites the third nucleus. When neurons of the third nucleus reach their threshold, these neurons strongly inhibit the activity of the first and the second nuclei. The balance of the weak inbound projections and strong out-bound projections of the nucleus third defined the duration of the output neuronal activity of the OM.

Results

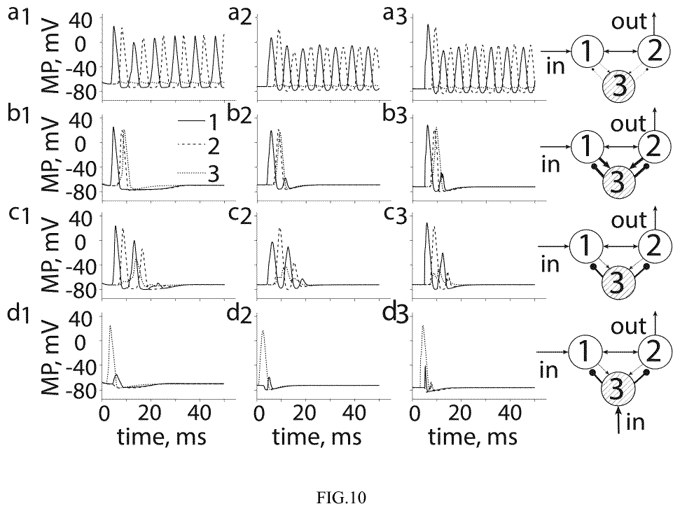

With the NEURON neuro-simulator the Hodgkin-Huxley model (see Hodgkin, A. L., Huxley, A. F.: A quantitative description of membrane current and its application to conduction and excitation in nerve. The Journal of Physiology 117(4), 500-544 (1952)) of a neuron was used with the following parameters: the membrane capacitance is 1 μf/cm², the resistivity is 100n/cm, sodium and potassium channels used with 0:25/cm² and 0:045/cm² conductance (see Dougherty, K., Kiehn, O.: Firing and Cellular Properties of V2a Interneurons in the Rodent Spinal Cord. The Journal of neuroscience: the official journal of the Society for Neuroscience 30, 24-37 (2010)). The model is multi-compartment and consists of soma, dendrites, and axon, each part is presented as one compartment. The diameters of neurons' soma are in the range from 3 μm to 8 μm (see Chen, S., Yang, G., Zhu, Y., Liu, Z., Wang, W., Wei, J., Li, K., Wu, J., Chen, Z., Li, Y., Mu, S., OuYang, L., Lei, W.: A Comparative Study of Three Interneuron Types in the Rat Spinal Cord. PLOS ONE 11(9), e0162969 (2016)). The OM is combined of two main components: two nuclei that trigger each other thus generate extended activity (FIG. 1B) and three nuclei that form the negative feedback (FIG. 1c) as well as divergence (FIG. 1d) and convergence (FIG. 1e). Each nucleus contains 50 neurons with synapses ranging from 30 to 50 for each projection. The balancing between excitatory and inhibitory weights provides a variety of spiking activity (FIG. 10). The OM produces a sequence of spikes with weak inhibitory projections and also weak excitatory to the 3rd nucleus (FIG. 10a). The frequency of the output signals from the 2nd nucleus approximately 120 Hz while the third nucleus is in the subthreshold state. The activity continues 50 ms and does not decrease. When inhibitory and excitatory projections are strong, the neuronal activity is dramatically reduced because the 1st nucleus triggers 2nd, which produces one output signal, and third nuclei simultaneously then 3rd powerfully inhibits both excitatory nuclei (shown FIG. 10b). The number of spikes is adjusted by the difference between excitatory and inhibitory weights when connections between excitatory nuclei (1 and 2) and the 3rd are weak. Whereas inhibitory projections are powerful, an output nucleus produces two averaged peaks from 7 ms to 20 ms. The excitatory nuclei gradually increases the potential of third nuclei. When it exceeds a threshold, the third nuclei is activated and inhibits first and second nuclei (shown FIG. 10c). The duration of the output neuronal activity also depends on external signals which trigger the third nucleus. If the weight of the inhibitory connection is sufficient, the output activity stops (FIG. 10d).

The second series of experiments (FIG. 10 a2, b2, c2, d2) is dedicated to tests of the OM in the GRAS simulator. GRAS was created for the GPU processing of bio-plausible neuronal networks using C++ programming language and Nvidia CUDA technology. With GRAS neurosimulator, the Hodgkin-Huxley model of a neuron was used with following parameters: the membrane capacitance is 1 μF/cm², the resistance is 100 Ω/cm. The model is one compartment.

The third series of experiments (FIG. 10 a3, b3, c3, d3) is dedicated to tests of the OM in the NEST neurosimulator (see Jordan, J., Ippen, T., Helias, M., Kitayama, I., Sato, M., Igarashi, J., Diesmann, M., Kunkel, S.: Extremely scalable spiking neuronal network simulation code: From laptops to exascale computers. Frontiers Neuroinformatics 12, 2 (2018)) simulator. The Hodgkin-Huxley model of a neuron with following parameters was used: the membrane capacitance is 1 ρf/cm², the resistance is 100 Ω/cm, sodium and potassium channels used with the default conductance. The model is one compartment.

Discussion

The comparison of the first mode with weak projections between nuclei 1, 2, 3 (FIG. 10 a1, a2) indicates the lower synaptic delay used in GRAS and higher amplitudes of spikes in NEURON simulator. The second mode with highest weight of projections between nuclei 1, 2 and 3 (FIG. 10 b1, b2) shows the shorter spike and refractory periods used in GRAS though similarly to the NEURON simulation both 1 and 2 nuclei produce one spike before the 3^{rd} nuclei intensively inhibits them.

In a third mode (FIG. 10 c1, c2) when weights are between first and second modes NEURON simulation nucleus 3 generates inhibiting spiking activity later than in the second mode similar to GRAS.

Overall there is higher similarity of NEST and GRAS simulation results. The comparison of the OM activity with low weights of nuclei 1, 2, 3 (FIG. 10 a1, a2, a3) indicates higher spiking rate 7 for NEST and 7 for GRAS with 6 for NEURON due to shorter refractory period during whole simulation 50 ms. The highest weights used in the second experiment (FIG. 10 b1, b2, b3) indicates the results of NEST close to the GRAS simulator with same equidistant spikes during 15 ms that is explained by the close models with relatively less details than in NEURON simulator. FIG. 10 c1, c2, c3 represent results of the simulation of OM with weights in the middle of the range between the first and second experiments where the short series of spikes 2 in NEURON and 3 in GRAS are observed, NEST simulators with decreased amplitude during 20 ms. The projection to the nucleus 3 increases the inhibitory influence of nuclei 1 and 2 and results with low amplitude subthreshold neuronal activity (FIG. 10 d1, d2, d3).

Conclusion

This Example 1 shows that the OM, which is more complex than previously proposed models can be used as the basic building block for complex neural circuits, for example for a mammalian pattern generator. The proposed organization of OM using previously described four motifs of the central nervous system: rhythmic excitation, feedback inhibition, divergence, and convergence has been supported by the data (see FIG. 10). The bio-plausible simulation results using three neurosimulators have been demonstrated. Results of simulation indicate that the proposed OM can be used in networks responsible for generation of neuronal activity, i.e. for simulation of locomotion.

### Example #2 (Even Simpler Real-time Model of Neuron)

Abstract

This Example #2 shows the simplified model of a neuron for the real-time processing in embedded bio-compatible devices. The bio-compatible model of a neuron includes works of Izhikevich (see Izhikevich, E.: Simple model of spiking neurons. IEEE Transactions on Neural Networks 14(6), 1569-1572 (2003)) and Rozenblatt (see Rosenblatt, F.: The perceptron: a probabilistic model for information storage and organization in the brain. Psychological review 65(6), 386 (1958)) and presents the block diagram and main principles of signals processing and learnings. The other simplified model of the neuron using other close to proposed models of leaky integrate and fire (LIF) and Izhikevich (IZH) have been validated. Moreover, the present simplified model shows better performance than IZH and LIF models and bio-compatibility from the temporal parameters perspective.

Introduction

There are several previously published models of neuron (see, e.g., Hodgkin, A. L., Huxley, A. F.: A quantitative description of membrane current and its application to conduction and excitation in nerve. The Journal of Physiology 117(4), 500{544 (1952); McCulloch, W. S., Pitts, W.: A logical calculus of the ideas immanent in nervous activity. The bulletin of mathematical biophysics 5(4), 115-133 (1943)). The database of neuron models called “model DB” of the Yale University now has several thousand of models published (see McDougal, R. A., Morse, T. M., Carnevale, T., Marenco, L., Wang, R., Migliore, M., Miller, P. L., Shepherd, G. M., Hines, M. L.: Twenty years of ModelDB and beyond: building essential modeling tools for the future of neuroscience. Journal of Computational. Neuroscience 42(1), 1-10 (2017)). One of critical problems of a bio-plausible neurosimulation is the time of computation. This is especially relevant taking into account that main impact over computation time is performed by synapses and neuronal models. In one instance, for example, autonomous robot (see Lobov, S., Kazantsev, V., Makarov, V. A.: Spiking neurons as universal building blocks for hybrid systems. Advanced Science Letters 22(10), 2633-2637 (2016)) or wearable neuroprosthesis (see, e.g., Deska-Gauthier, D., Zhang, Y.: The functional diversity of spinal interneurons and locomotor control. Current Opinion in Physiology 8, 99-108 (2019); Gill, M. L., Grahn, P. J., Calvert, J. S., Linde, M. B., Lavrov, I. A., Strommen, J. A., Beck, L. A., Sayenko, D. G., Straaten, M. G. V., Drubach, D. I., Veith, D. D., Thoreson, A R., Lopez, C., Gerasimenko, Y. P., Edgerton, V. R., Lee, K. H., Zhao, K. D.: Neuromodulation of lumbosacral spinal networks enables independent stepping after complete paraplegia. Nature Medicine (2018); Wagner, F. B., Mignardot, J. B., Go-Mignardot, C. G. L., Demesmaeker, R., Komi, S., Capogrosso, M., Rowald, A., Senez, I., Caban, M., Pirondini, E., Vat, M., McCracken, L. A., Heimgartner, R., Fodor, I., Watrin, A., Seguin, P., Paoles, E., Keybus, K. V. D., Eberle, G., Schurch, B., Pralong, E., Becce, F., Prior, J., Buse, N., Buschman, R., Neufeld, E., Kuster, N., Carda, S., Zitzewitz, J.v., Delattre, V., Denison, T., Lambert, H., Minassian, K., Bloch, J., Courtine, G.: Targeted neurotechnology restores walking in humans with spinal cord injury. Nature 563(7729), 65 (2018)), a real-time processing for the topology of thousands of neurons and hundreds of thousands of synapses on a single board computer with limited weight and usually no connection to the network is required.

A simplified model of a neuron corresponding to the following requirements is presented: a real-time processing on the one-board computer taking in account bio-plausible spike times, refractory period, inhibitory influence and threshold-based processing of the inbound spikes.

Models Comparison

For the performance comparison three most closely related models of a spiking neuron have been used: LIF model, IZH model, and a simplified digital neuron (SDN), proposed below. The three models are close from the performance perspective, IZH and SDN model were specifically designed to save the computation time.

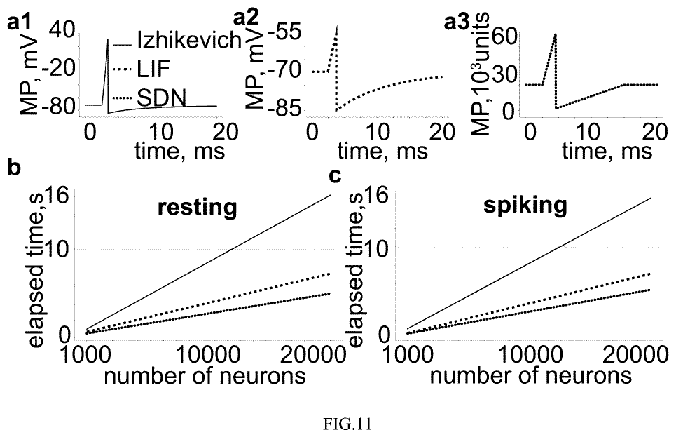

The IZH is a simplification of the Hodgkin-Huxley model and has bio-plausible parameters, such as time and amplitude of spike (FIG. 11 a1), but the maximal spike amplitude is used as threshold. This way, the width of a spike could be large (for example, 5 ms) in case of low weights of input synapses. In addition, IZH model can work in different modes depending on the type of neuron.

The LIF model uses the input current to charge the capacitor to update the membrane potential. This model is considered simpler than the IZH model. Parameters of the LIF model such as spiking rate or refractory period correspond to parameters of a biological neuron (FIG. 11 a2). For the SDN model, the temporal parameters are most important factor for the real-time processing compare to amplitude that was used as unit pulses to reduce the calculation time on multiplication of a synaptic weight to corresponding spike amplitude. Thus, in the SDN model the bio-plausible refractory period and spike duration for the real-time processing neuron is used. In the SDN model the shape of the spike is similar to the LIF model spike, but the refractory period curve to the resting state is not smooth (FIG. 11 a3). The current is not used to calculate the level (membrane potential) in the SDN model. Instead, the current is represented by the sum of projection weights, leakage, and noise to increase the performance of the model.

SDN

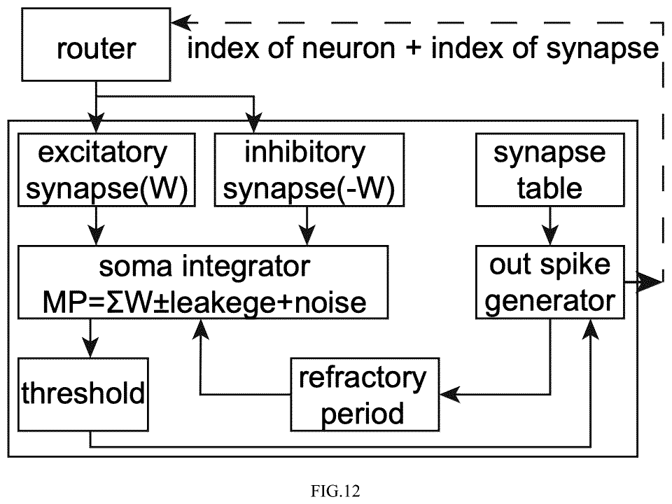

A simulation of the simplified neuronal model capable of real-time processing in the topology of thousands of neurons, as the time of calculation is critical problem of a bio-plausible neuron models. Here, the simplified digital neuron (SDN) model, which corresponds to the following requirements: real-time processing on the one-board computer, taking in account bio-plausible spike times, refractory period, inhibitory influence, and threshold-based processing of the inbound spikes is provided. As shown on FIG. 12, the signals coming to the excitatory synapses increase the probability of the neuron to generate the outbound spike, while inputs to the inhibitory synapses spike decreases. The inputs can not influence the spike level (membrane potential) during the refractory period. In the SDN model the following assumptions to optimize the code of the neuronal model for the real-time processing of several thousands of neurons and hundreds of thousands of synapses are taken into account:
1. Only the spinal cord biological neurons (motor neurons and interneurons) are considered;
2. The amplitude of spikes for the effectiveness of the computation is excluded;
3. The influence of inhibition decrease the probability of neurons to generate the outbound spike; and
4. For the temporal parameters of the outbound neuronal response, bio-plausible spike and refractory period duration has been utilized.

L=ΣW±leakage+noise (1)

For purposes of the computation in the real-time, the simplified equation for the level that resembles the membrane potential (Eq. 1 above) has been provided, where the level is the sum of projection weights, leakage, and noise. When the level is over the specified threshold the model produces a signal that is represented by unit pulse. The block diagram of the SDN is shown in FIG. 12. The input unit signals (spikes) are processed by the router that transfers them to the corresponding neuron and its synapse. All synapses of an SDN store their weight because each spike is represented as “1” there is no need to multiply a synaptic weight to input. The dynamics of the soma is maintained via the leakage parameter that represents the sodium/potassium pumps and leakage of potassium through the semi-permissible membrane. It has been discovered that noise is important even in this simplified model for the reason of stochasticity of all neuronal channels even in simple circuitry in the spinal cord. Leakage and noise parameters are set for each neuron individually. The threshold function triggers the output spike generator with a preset duration when the level reaches the threshold value. The refractory period is implemented as negative feedback to the soma integrator that decreases the level to the present value for the set period individually to each cell. The synapse table stores the neurons and their synapses with axonal and synaptic delays to address the output spike through the router.

To reduce RAM consumption and decrease calculation time, C++ unsigned short (uint16_t) type (2 bytes) for level variable has been used because floating point operations are slower than integer operations (see, e.g., Limare, N.: Integer and Floating-Point Arithmetic Speed vs Precision (2014)). The use of unsigned short allows to balance between possible states of the level (in abstract unsigned units from 0 to 65 535) and saving to the RAM. Thus, it is possible to allocate extra SDN neurons at a rate that is twice more than LIF and four times more than the IZH models (4 and 8 times, respectively if double is used). This can be important for single board computers implementation of the network.

The SDN model allows for low computation cost for the individual neuron parameters variability while keeping the processing of thousands of neurons with hundreds of thousands of synapses calculation in the real-time.

Results

To compare three models the isolated computer with the parallel (OpenMP) implementation of same neural network size with different neuronal models on CPU was used (Intel® Xeon® Processor E5-2650 v2 (8 cores, 2:60 GHz)) with 96 GB RAM. During the simulation, all neuron models have constant parameters except for the membrane potential (and U m of the IZH model). Those parameters were implemented as arrays. Two modes of the simulation (resting and spiking that affects the state of neurons) was tested. During the resting mode of the simulation there is no input current in any neuron. In contrast, during the spiking mode, the spiking activity is triggered of all neurons every 7.5 ms (the period of spike plus refractory period to guarantee reaching of the resting potential). In order to minimize the side effects over the computing time measurement of a neuron mode, no synapses, no spike or potential recorders were used.

Simulation includes 10 tests per neuron, the network size being from 1,000 to 20,000 neurons with step size of 1,000. Total simulations for one model is 200.

Resulting data is shown in FIG. 11 and presented as a trend-line (using the polyfit function of NumPy package). In the resting mode (FIG. 11b), SDN and LIF models are faster than real-time with 20,000 neurons. The IZH model showed timing below the real-time threshold if the number of neurons is less than 13,000. In the spiking mode (FIG. 11c) SDN and LIF fit the real-time requirement while IZH is above the mark. All three tested models have linear time dependencies with different slopes in FIGS. 11b and c, due to additional arithmetic operations and different number of variables.

Conclusion

The lightweight neuronal model for real-time computing with, for example, limited hardware, with a wearable device has been provided. The performance comparison of the IZH and LIF models in two modes (resting and spiking) indicated the better performance of the SDN model.

The foregoing detailed description of the embodiments is used to further clearly describe the features and spirit of the present invention. The foregoing description for each embodiment is not intended to limit the scope of the present invention. All kinds of modifications made to the foregoing embodiments and equivalent arrangements should fall within the protected scope of the present invention. Hence, the scope of the present invention should be explained most widely according to the claims described thereafter in connection with the detailed description, and should cover all the possibly equivalent variations and equivalent arrangements.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method for reconstruction of a neuronal topology responsible for activation of motor functions in limbs, the method comprising:
detecting a motor evoked response pattern in correlation with input stimulation parameters and sensory modulation;
analyzing the functional motor evoked response pattern for a neuronal response pattern, the neuronal response pattern is determined based on a plurality of peaks, each peak having a maximum value and a minimum value, and each peak having amplitude and time; and
reconstructing the neuronal circuit topology by comparing the neuronal response pattern with a known or hypothetical (based on results) neuronal topology that forms an output signal to activate motor function in limbs.

2. The method of claim 1 further comprising simulating the neuronal response pattern using a neuronal activity simulator.

3. The method of claim 2, wherein the neuronal activity simulator is a GRAS simulator.

4. The method of claim 3, wherein the GRAS simulator uses a simplified digital neuron (SDN) model.

5. The method of claim 1, wherein the neuronal response pattern is detected based on amplitude, frequency and duty cycle of an isolated neuronal circuitry.

6. The method of claim 1, wherein an oscillator motif (OM) is used for generating the neuronal response pattern, wherein the OM includes reciprocal excitation component and feedback inhibition component.

7. The method of claim 1, wherein a multi-level spinal locomotor circuit (mSLC) model is used for generating the neuronal response pattern, the mSLC model comprises a monosynaptic layer and a polysynaptic layer.

8. A neuromorphic prosthesis system for facilitating sensorimotor function in a subject in need thereof, the system comprising:
a plurality of stimulators having one or more channels configured to provide an electrical stimulation of muscles groups, peripheral nerves, plexuses and/or spinal cord;
a plurality of sensors configured to detect and communicate a continues or cyclic distribution of a body weight, angle joint motions, kinetics and/or electrophysiological parameters data;
a plurality of controllers configured to receive a combined data from the plurality of sensors and configured to process the combined data and communicate the combined data to an fMEP-based artificial circuitry device, the fMEP-based artificial circuitry device configured for implementation of a reconstruction of topology of central pattern generator (CPG), wherein the fMEP-based artificial circuitry device applies a bio-plausible neuronal topology for a motor function pattern and coordinates application of electrical signals via the plurality of stimulators, compatible with a biological neuronal stimulation, to the subject in need thereof to perform the motor function pattern.

9. The system of claim 8, wherein the fMEP-based artificial circuitry device comprises a synchronized set of one board digital computers.

10. The system of claim 8, wherein the fMEP-based artificial circuitry device comprises spiking analog schematics implementing a central pattern generator of a segment of a spinal cord topology or an application-specific integrated circuit (ASIC) chip.

11. The system of claim 9 further comprising:
a universal asynchronous receiver-transmitter (UART) serial port; and
a digital-to-analog converter (DAC), wherein DAC is configured to convert voltage signals received the plurality of stimulators into current signals.

12. The system of claim 10 further comprising:
a digital-to-analog converter (DAC), wherein DAC is configured to convert voltage signals received by the plurality of stimulators into current signals.

13. The system of claim 8, wherein the plurality of sensors comprises a pressure sensor positioned around an insole area and a flex sensor position around an ankle area of the subject.

14. The system of claim 8, wherein the plurality of stimulators comprise a back stimulator, a left ankle stimulator, a right ankle stimulator, a right hip stimulator, and a left hip stimulator.

15. A method for activation of a motor function in limbs of a subject in need thereof, the method comprising:
providing a plurality of stimulators having one or more channels configured to provide electrical stimulation of muscle groups, peripheral nerves, plexuses, or spinal cord; providing a plurality of sensors configured to detect and communicate a continuous or cyclic distribution of body weight, angle joint motions, kinetics, and electrophysiological parameters data from the plurality of sensors;
providing a plurality of controllers configured to receive a combined data from the plurality of sensors and configured to process the combined data and communicate the combined data to an fMEP-based artificial circuitry device, the fMEP-based artificial circuitry device configured for implementation of a reconstruction of topology of central pattern generator (CPG); and
stimulating the subject in need thereof to perform the motor function via electrical signals applied by the plurality of stimulators to the subject, wherein the fMEP-based artificial circuitry device applies a bio-plausible neuronal topology for the motor function pattern and coordinates application of the electrical signals.

16. The method of claim 15, wherein the bio-plausible neuronal topology is determined by a method comprising:
detecting a neuronal response pattern in correlation with input stimulation parameters;
analyzing the neuronal response pattern, wherein the neuronal response pattern is determined based on a plurality of peaks, each peak having a maximum value and a minimum value, and each peak having amplitude and time; and
reconstructing the neuronal circuit topology by comparing the neuronal response pattern with a known neuronal topology that forms an output signal to activate motor function in limbs.

17. The method of claim 16 further comprising simulating the neuronal response pattern using a neuronal activity simulator.

18. The method of claim 17, wherein the neuronal activity simulator is a GRAS simulator.

19. The method of claim 18, wherein the GRAS simulator uses a Even Simpler Real-Time Model of Neuron (ESRN) model.

20. The method of claim 16, wherein the neuronal response pattern is detected based on amplitude, frequency and duty cycle of an isolated neuronal circuitry.

21. The method of claim 16, wherein a multi-level spinal locomotor circuit (mSLC) model is used for detecting the neuronal response pattern, the mSLC model comprises a monosynaptic layer and a polysynaptic layer.
